(19) United States
(12) Patent Application Publication
(10) Pub. No.: US 2026/0014387 A1
(43) Pub. Date: Jan. 15, 2026

(54) LED Face Mask

(71) Applicant: Tech Activated Beauty, LLC, Juno Beach (FL)

(72) Inventors: Lloyd Nelson, Largo, FL (US); Toni Battaglia, Juno Beach, FL (US)

(21) Appl. No.: 18/766,751

(22) Filed: Jul. 9, 2024

(51) Int. Cl. A61N 5/06; H01R 13/62

(52) U.S. Cl. CPC A61N 5/0616; A61N 2005/0647; A61N 2005/0652; H01R 13/6205; H01R 2201/12

(57) ABSTRACT

The modular led light therapy mask addresses the shortcomings in the prior art by offering a modular LED light therapy mask that enables targeted treatments for various facial areas, with the flexibility to treat the upper face, lower face, or full face depending on the user's requirements. The device's innovative use of conductive magnetic connections to power and control the lower unit sets it apart from existing devices. The modular led light therapy mask for targeted facial treatments provides targeted treatments for various facial areas, including the reduction of wrinkles and fine lines, treatment of acne, lip plumping, and hairline follicle health enhancement. The device uses spectrum-specific LED light therapy to achieve its intended purposes.

### FIELD

This invention relates to the field of skin treatment and more particularly to a system for selectively treating certain areas of the skin of the face with differing light frequencies depending on the desired result.

### BACKGROUND

Light therapy is used for various medical and cosmetic purposes. The use of light-emitting diodes (LEDs) in light therapy has gained significant attention in recent years due to their non-invasive nature, safety, and efficacy in treating a variety of skin conditions.

Today, LED light therapy is used in dermatology clinics, spas, and at-home devices. Professional-grade devices typically offer higher light intensity and targeted treatments, while at-home devices provide convenience and lower-intensity treatments for maintenance and general skin health.

Despite the advancements in LED light therapy technology, there remains a need for devices that offer targeted treatments for specific facial areas and allow for customization based on individual needs and preferences. Additionally, there is a growing demand for hands-free, wireless devices that provide a comfortable and convenient user experience.

### SUMMARY

The modular led light therapy mask addresses the shortcomings in the prior art by offering a modular LED light therapy mask that enables targeted treatments for various facial areas, with the flexibility to treat the upper face, lower face, or full face depending on the user's requirements. The device's innovative use of conductive magnetic connections to power and control the lower unit sets it apart from existing devices.

The modular led light therapy mask for targeted facial treatments provides targeted treatments for various facial areas, including the reduction of wrinkles and fine lines, treatment of acne, lip plumping, and hairline follicle health enhancement. The device uses spectrum-specific LED light therapy to achieve its intended purposes.

The mask is composed of two main parts: a structured upper unit and a soft, pliable lower unit. The upper unit, or upper half, houses the power and control functions and is adapted to treat the upper half of the face. The lower unit, or lower half, connects magnetically to the upper unit, allowing for full-face treatment when desired. This modular design enables users to customize their treatments based on their needs and budget, as they can opt for either upper face or full-face treatments, detaching the lower unit from the upper unit when only upper facial treatment is desired.

The device is designed to be wireless and hands-free, ensuring user convenience. The upper unit features a structured base made of plastic and silicone, shaped to match the shape of the typical upper half of a user's face. Acceptable materials include polycarbonate or ABS plastic.

The lower unit, made of soft, pliable silicone, follows the contours of the upper unit when attached via the magnetic connections. The magnetic connections create physical connections between the upper and lower units, while also carrying electrical power and control signals between the upper and lower units. The flexible lower unit uses flexible circuit boards and wiring to carry power and signals to the multiple LEDs, maintaining connectivity as it is shaped to match the contours of the user's face.

The mask utilizes medical-grade, spectrum-specific LEDs that convert 85% or more of incoming electrical power to light output, minimizing heat generation. The materials used in the device, such as ABS, polycarbonate, and medical-grade TPE silicone, are FDA-approved and meet biocompatibility testing requirements.

The LEDs are placed strategically to perform specific facial treatments in the following areas:

For treatment of the hair line, 830-880 nm IR and 625-660 nm red dual chip LEDs increase blood circulation to the hair follicles for improved follicle health. This helps a user compensate for damage that results from hair styling.

For treatment of the lips, 830-850 nm and 630-660 nm dual chip LEDs are sued to improve circulation and plumpness of the lips.

For treatment of acne, 415 nm and 630 nm dual chip LEDs are placed in areas of the mask that treat the forehead, cheeks, nose, jawline and chin.

For anti-aging treatment, 630/830 nm and 605/660 mm dual chip LEDs are placed in areas of the mask that treat the forehead, cheeks, jawline, chin and under-eye areas.

In the preferred embodiment, the LEDs integrated into the device include:
Twenty-eight (830 nm/630 nm) dual-chip LEDs placed around the mouth area;
Ten dual-chip LEDs placed (830 nm/660 nm) on the hairline;
Fourteen dual-chip LEDs placed around each eye opening, specifically eight dual-chip LEDs (830 nm/630 nm) to the left and right of each eye opening, and six dual-chip LEDs under the eye opening;
Eighteen dual-chip LEDs (830 nm/630 nm) dispersed throughout the remaining area of the mask;
Eighteen dual-chip LEDs (660 nm/605 nm) dispersed throughout the remaining area of the mask; and
Thirty-six dual-chip LEDs (415 nm/630 nm) dispersed throughout the forehead, nose, chin, jawbone and cheek areas.

The control electronics include wireless communication protocols, including Bluetooth and similar, to enable control, monitoring, and reporting using devices such as a mobile phone.

To operate the device, it is first fully charged. This is accomplished by plugging the unit into a source of power, preferably via a common connector, such as a USB-C connector. The internal battery is charged, storing power for later use.

When charged, the user can unplug the device, then deciding whether to attach the lower unit to the upper unit.

The user places the device onto their face and toggles the power button to cycle through the treatment scenarios. Treatment scenarios include acne treatment and wrinkle reduction.

After the user has selected a treatment plan, the unit begins the associated program. Each program includes the activation of particular LEDs to create certain light frequencies in the selected physical locations that correspond to the areas to be treated.

The device shuts off automatically when the treatment is complete.

The modular design, targeted treatment capabilities, and the innovative use of conductive magnetic connections to power and control the lower unit set this LED light therapy mask apart from prior art in the field.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates a first isometric view of the modular led light therapy mask.

FIG. 2 illustrates a second isometric view of the modular led light therapy mask.

FIG. 3 illustrates a front view of the modular led light therapy mask.

FIG. 4 illustrates a back view of the modular led light therapy mask.

FIG. 5 illustrates a left view of the modular led light therapy mask.

FIG. 6 illustrates a right view of the modular led light therapy mask.

FIG. 7 illustrates a third isometric view of the modular led light therapy mask.

FIG. 8 illustrates a first view of the magnetic connector of the modular led light therapy mask.

FIG. 9 illustrates a second view of the magnetic connector of the modular led light therapy mask.

FIG. 10 illustrates a view of the upper section of the magnetic connector of the modular led light therapy mask.

FIG. 11 illustrates a view of the lower section of the magnetic connector of the modular led light therapy mask.

FIG. 12 illustrates a view of the device with all LEDs of the modular led light therapy mask.

FIG. 13 illustrates a first view of the PCBs showing treatment areas of the modular led light therapy mask.

FIG. 14 illustrates a second view of the PCBs showing active LEDs for acne treatment of the modular led light therapy mask.

FIG. 15 illustrates a third view of the PCBs showing active LEDs for acne and anti-aging treatments of the modular led light therapy mask.

FIG. 16 illustrates a fourth view of the PCBs showing active LEDs for anti-aging treatment of the modular led light therapy mask.

### DETAILED DESCRIPTION

Reference will now be made in detail to the presently preferred embodiments of the invention, examples of which are illustrated in the accompanying drawings. Throughout the following detailed description, the same reference numerals refer to the same elements in all figures.

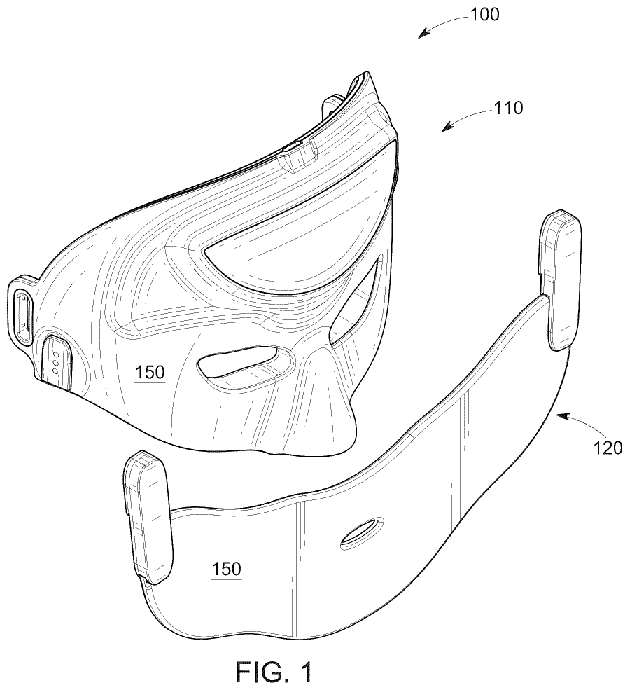

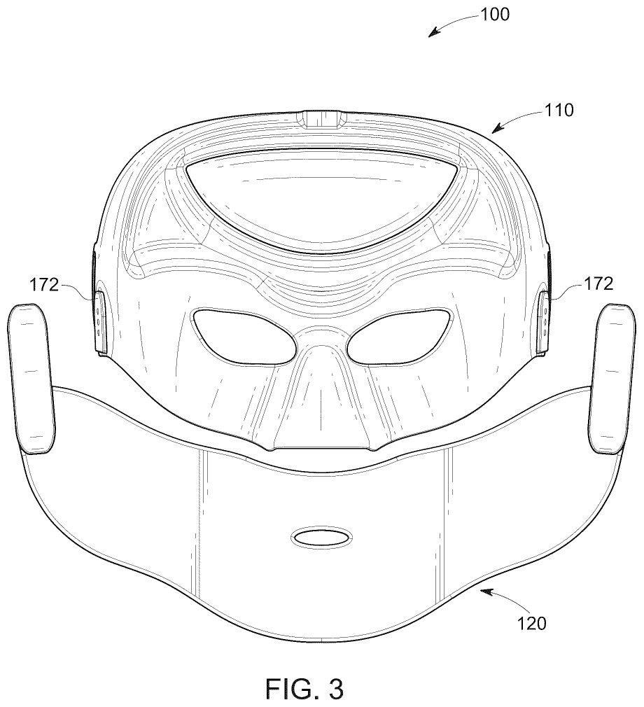

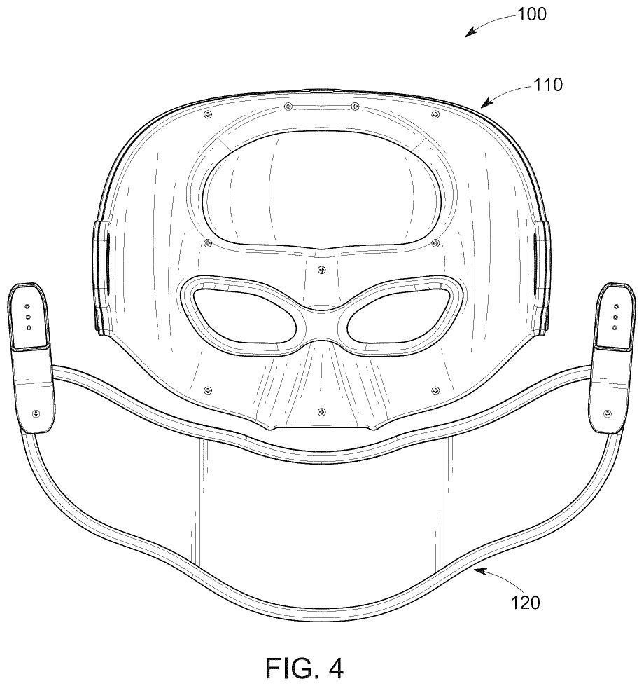

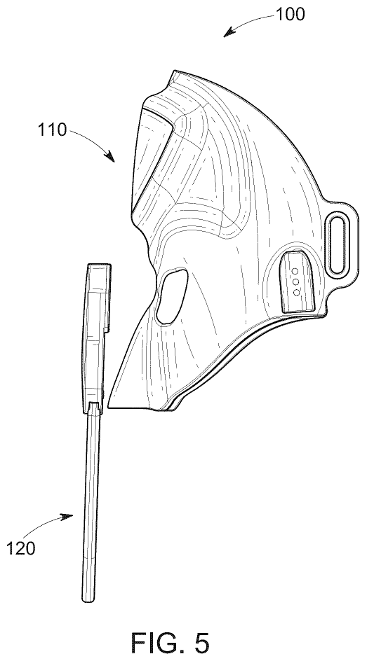

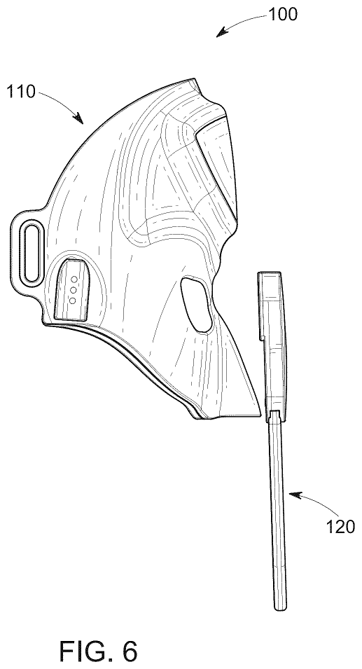

Referring to FIGS. 1-6, various view of the modular led light therapy mask are shown.

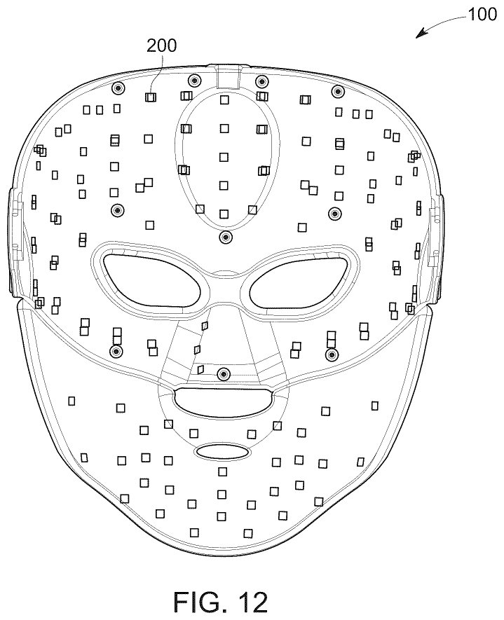

The modular led light therapy mask for targeted facial treatments 100 includes the upper unit 110 and the lower unit 120, each with an opaque outer cover 150 and a translucent inner cover 152. The LEDs 200 (see FIG. 12) shine light through the translucent inner cover 152 to treat the user's skin.

The magnetic connectors 170 that affix the upper unit 110 to the lower unit 120 include a mating magnetic connector protrusion 172 and a magnetic connector recess 174.

The lower unit 120 is constructed from flexible materials, allowing it to bend around curvature of the lower half of the user's face. The lower unit 120 is shown in its flat storage position, ready to be curved and affixed to the upper unit 110.

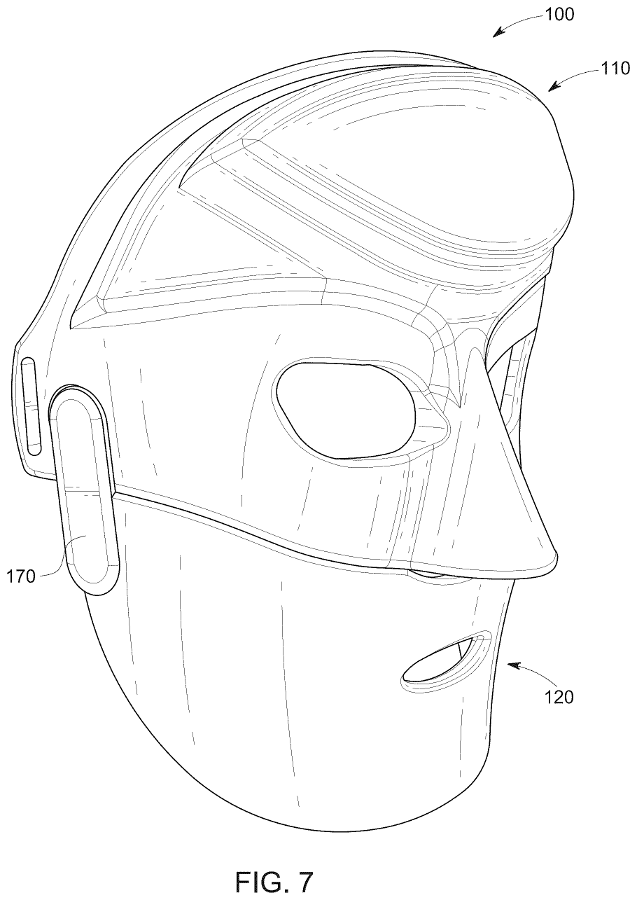

Referring to FIG. 7, a third isometric view of the modular led light therapy mask is shown.

The lower unit 120 is now shown curved to match curved shape of the upper unit 110. Magnetic connectors 170 have been joined across the upper unit 110 and lower unit 120, holding the two halves of the device together.

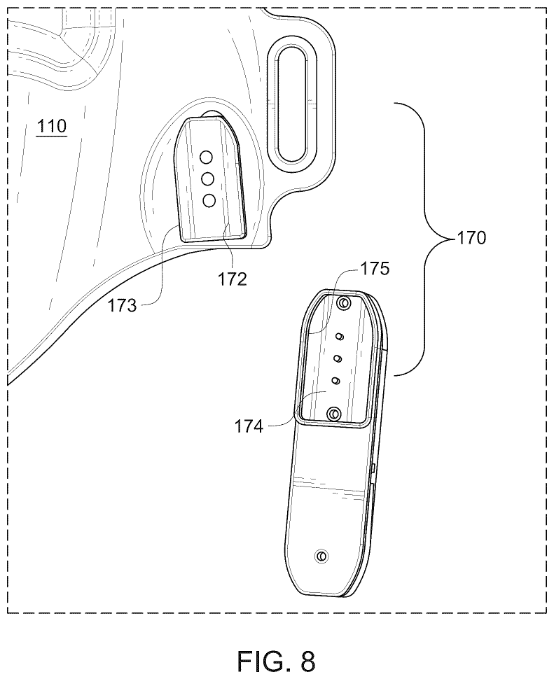

Referring to FIG. 8, a first view of the magnetic connector of the modular led light therapy mask is shown.

The magnetic connector 170 is shown with magnetic connector protrusion 172 associated with the upper unit 110 and magnetic connector recess 174 associated with the lower unit 120 (see FIG. 1). To connect, the magnetic connector protrusion 172 fits within the magnetic connector recess 174, the outside perimeter 173 of the magnetic conductor protrusion 172 resting against the inside perimeter 175 of the magnetic connector recess 174, preventing rotation, sliding, and shifting of the lower unit 120 with respect to the upper unit 110.

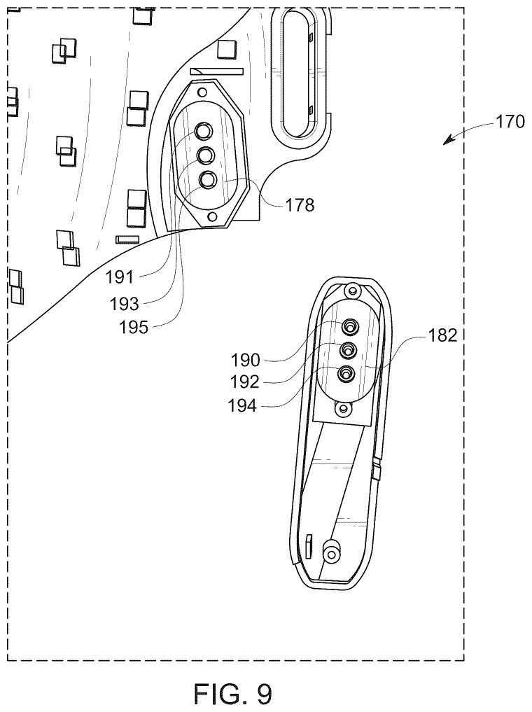

Referring to FIG. 9, a second view of the magnetic connector of the modular led light therapy mask is shown.

To carry electrical signals between the upper unit 110 and the lower unit 120 (see FIG. 7), the magnetic connectors 170 include power and signal pins. The grounding pin 190 mates with the grounding socket 191, the first signal pin 192 mates with the first signal socket 193, the second signal pin 194 mates with the second signal socket 195.

The upper unit magnet 178 attracts the lower unit magnet 182, drawing the magnetic connectors 170 toward each other.

Referring to FIG. 10, a view of the upper section of the magnetic connector of the modular led light therapy mask is shown.

The upper unit connector PCB 196 is shown with grounding socket 191, first signal socket 193, and second signal socket 195.

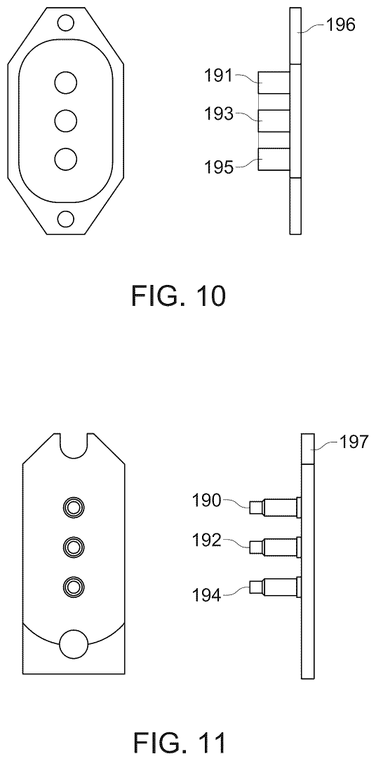

Referring to FIG. 11, a view of the lower section of the magnetic connector of the modular led light therapy mask is shown.

The lower unit connector PCB 197 is shown with grounding pin 190, first signal pin 192, and second signal pin 194.

Referring to FIG. 12, a view of the device with all LEDs of the modular led light therapy mask is shown.

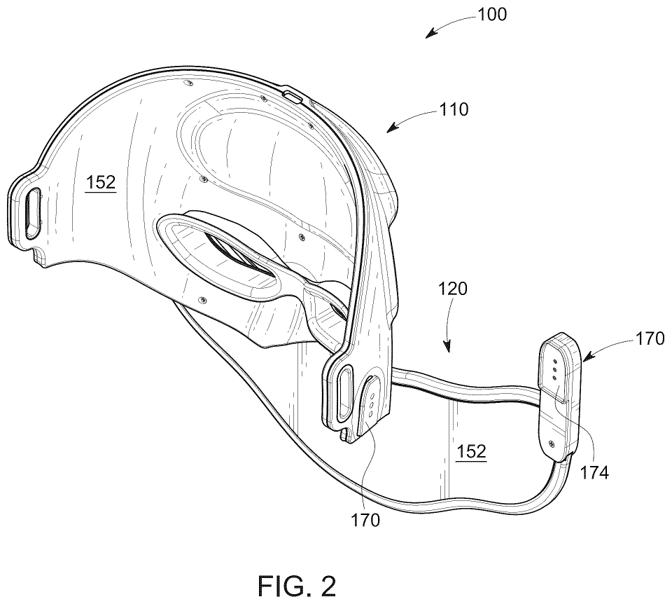

The modular led light therapy mask for targeted facial treatments 100 includes a multiplicity of LEDs 200 that face inward, toward the user. By hiding the translucent inner cover 152 (see FIG. 2), the LEDs 200 are visible.

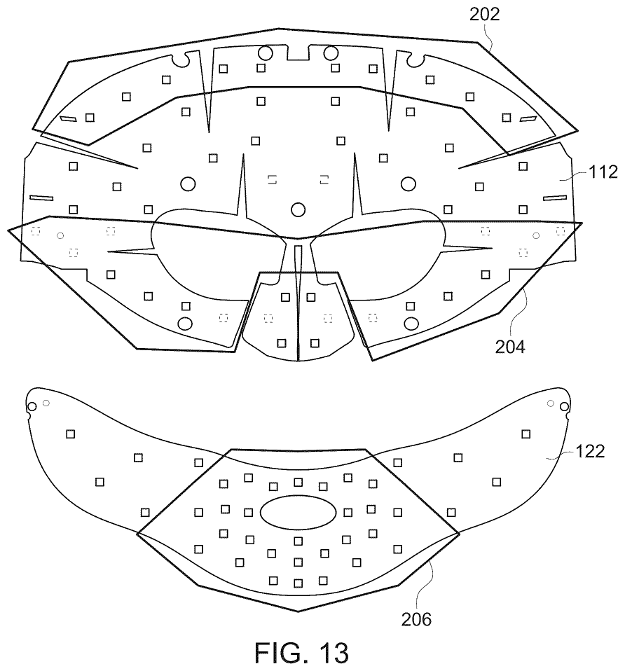

Referring to FIG. 13, a first view of the PCBs showing treatment areas of the modular led light therapy mask is shown.

The LEDs 200 are divided into different areas, or regions, across the upper unit PCB 112 and the lower unit PCB 122.

The preferred divisions include a hairline treatment region 202 across the upper section of the upper unit PCB 112, an eye treatment region 204 across the lower section of the upper unit PCB 112, and a lip treatment region 206 that is centrally located within the lower unit PCB 122.

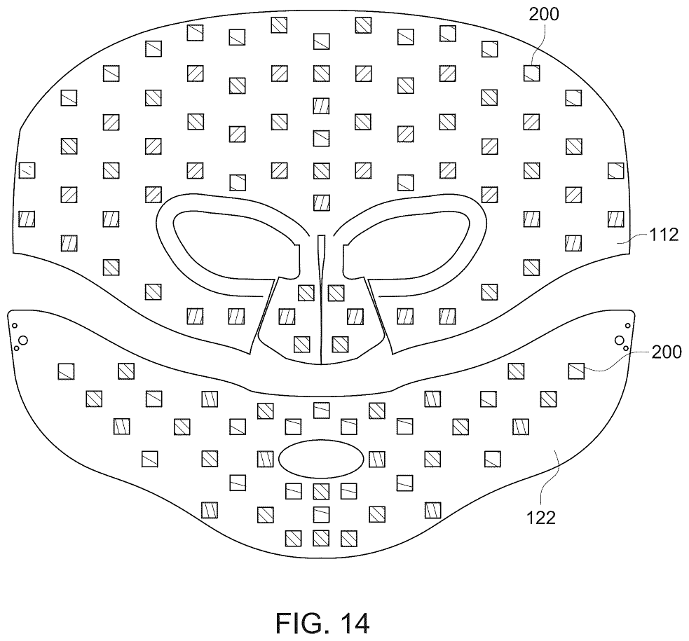

Referring to FIG. 14, a second view of the PCBs showing active LEDs for acne treatment of the modular led light therapy mask is shown.

The LEDs 200 that are shaded are active during the modes that include acne treatment and anti-aging treatment.

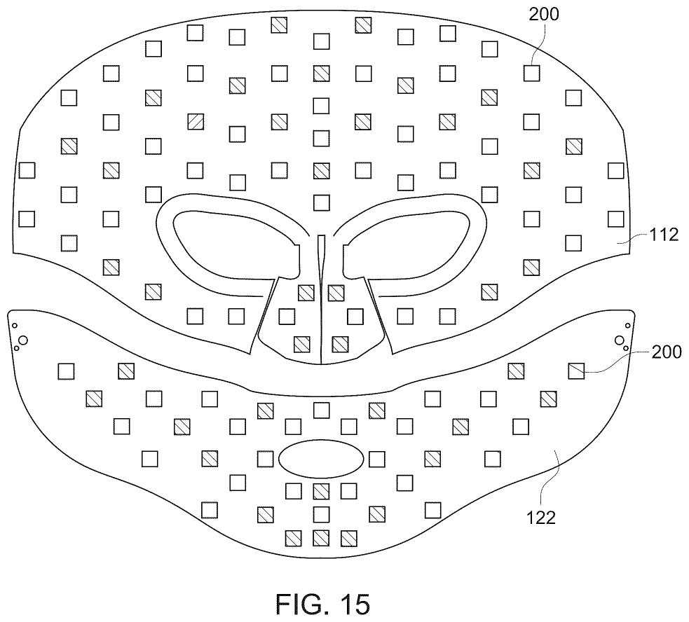

Referring to FIG. 15, a third view of the PCBs showing active LEDs for acne and anti-aging treatments of the modular led light therapy mask is shown.

The LEDs 200 that are shaded are active during the modes that include anti-aging treatment.

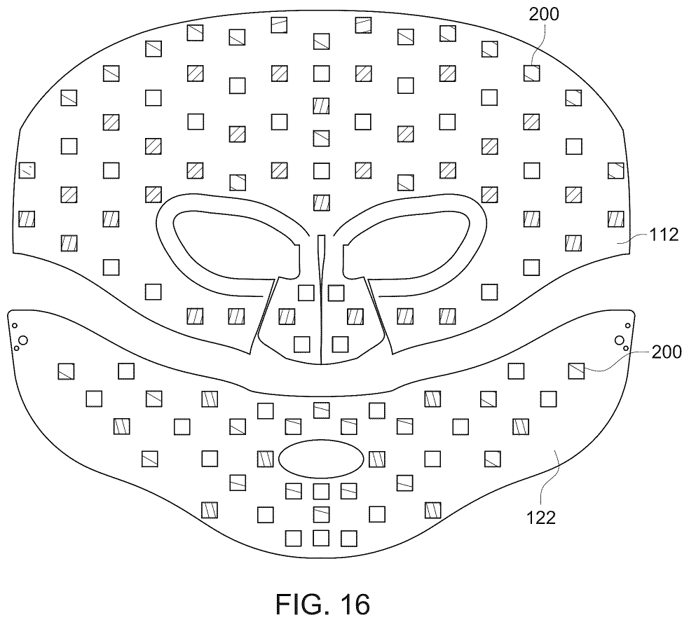

Referring to FIG. 16, a fourth view of the PCBs showing active LEDs for anti-aging treatment of the modular led light therapy mask is shown.

The LEDs 200 that are shaded are active during the modes that include acne treatment.

Equivalent elements can be substituted for the ones set forth above such that they perform in substantially the same manner in substantially the same way for achieving substantially the same result.

What is claimed is:

1. A mask for treatment of skin of a user's face, the mask comprising:
an upper unit;
the upper unit including an upper plurality of
integrated light emitting diodes for skin treatment;
and a lower unit;
the lower unit including a lower plurality of integrated light emitting diodes for skin treatment;
the lower unit detachable from the upper unit;
wherein the upper unit can operate alone to provide skin therapy, or the upper unit can operate in combination with the lower unit to provide skin therapy;
whereby a user can choose whether to wear the upper unit alone, or the upper unit and the lower unit together.

2. The mask for treatment of skin of a user's face of claim 1, further comprising:
a magnetic connector;
the magnetic connector removably joining the upper unit to the lower unit;
the magnetic connector carrying electrical power and signal from the upper unit to the lower unit;
whereby the user can detach the lower unit from the upper unit without tools.

3. The mask for treatment of skin of a user's face of claim 2, wherein the magnetic connector further comprises:
a magnetic connector protrusion that fits into a magnetic connector recess;
the magnetic connector protrusion preventing the magnetic connector recess sliding away, and therefore preventing the lower unit from sliding away from the upper unit.

4. The mask for treatment of skin of a user's face of claim 2, wherein the magnetic connector further comprises:
a grounding pin that mates with a grounding socket;
a first signal pin that mates with a first signal socket;
a second signal pin that mates with a second signal socket;
the magnetic connector therefore acting both as a physical connection and a connection that carries electrical signals and power between the upper unit and the lower unit.

5. The mask for treatment of skin of a user's face of claim 1, wherein:
the upper plurality of integrated light emitting diodes for skin treatment is divided into a hairline treatment region and an eye treatment region;
wherein the user can choose which regions to activate.

6. The mask for treatment of skin of a user's face of claim 1, wherein:
the lower plurality of integrated light emitting diodes for skin treatment includes a lip treatment region.

7. A facial skin treatment device, comprising:
an upper half comprising power and control electronics;
the upper half including an upper plurality of integrated light emitting diodes for skin treatment;
a removable lower half;
the removable lower half including a lower plurality of integrated light emitting diodes for skin treatment;
wherein the removable lower half allows a user to speak or eat while wearing the facial skin treatment device, and the upper half can be worn independently to treat skin of the upper half of a face.

8. The facial skin treatment device of claim 7, further comprising:
a magnetic connector;
the magnetic connector removably joining the upper half to the removable lower half;
the magnetic connector carrying electrical power and signal from the upper half to the removable lower half;
whereby the user can detach the upper half from the removable lower half without tools.

9. The facial skin treatment device of claim 8, the magnetic connector further comprising:
a magnetic connector protrusion that fits into a magnetic connector recess;
the magnetic connector protrusion preventing the magnetic connector recess sliding away, and therefore preventing the removable lower half from sliding away from the upper half.

10. The facial skin treatment device of claim 8, wherein the magnetic connector further comprises:
a grounding pin that mates with a grounding socket;
a first signal pin that mates with a first signal socket;
a second signal pin that mates with a second signal socket;
the magnetic connector therefore acting both as a physical connection and a connection that carries electrical signals and power between the upper half and the removable lower half.

11. The facial skin treatment device of claim 7, wherein:
the upper plurality of integrated light emitting diodes for skin treatment is divided into a hairline treatment region and an eye treatment region;
wherein the user can choose which regions to activate.

12. The facial skin treatment device of claim 7, wherein:
the lower plurality of integrated light emitting diodes for skin treatment includes a lip treatment region.

13. A skin treatment device that allows a user to decide whether to treat all of a face or only an upper half of the face, the skin treatment device comprising:
an upper half and a lower half;
the upper half and lower half each including plurality of light emitting diodes;
the upper half further including control and power mechanisms to operate the plurality of light emitting diodes;
the upper half removably attachable to the lower half via a magnetic connector;
the magnetic connector including a first half with a protrusion and a second half with a recess, interaction of the protrusion with the recess preventing the second half from sliding with respect to the first half, therefore preventing the lower half from sliding away from the upper half.

14. The skin treatment device of claim 13, wherein the upper half is operable separately, or in combination with the lower half.
